(19) United States
(12) Patent Application Publication
(10) Pub. No.: US 2026/0299320 A1
(43) Pub. Date: Oct. 1, 2026

(54) Visual Focus Device Kit

(71) Applicants: Jeffrey H. Farris, Hattiesburg, MS (US); William West, Houston, TX (US)

(72) Inventors: Jeffrey H. Farris, Hattiesburg, MS (US); William West, Houston, TX (US)

(21) Appl. No.: 19/704,362

(22) Filed: Jun. 10, 2026

Related U.S. Application Data
(63) Continuation-in-part of application No. 13/168,142, filed on Jun. 24, 2011.

(51) Int. Cl. G02C 7/16; A63B 69/00; A63B 102/18

(52) U.S. Cl. CPC G02C 7/16; A63B 69/0002; A63B 2069/0008; A63B 2102/18

(57) ABSTRACT

A visual aid device is disclosed. The visual aid device has opaque portions to block parts of and individual's vision. The visual aid device may provide a horizontal tunnel vision and be worn by athletes to block out peripheral visual distractions. The visual aid device may include upper and lower opaque portions disposed to block out peripheral vision along horizontal planes above and below the user's eyes. The visual aid device may be configured with an inner opaque portion for one eye to reduce the effects of double vision. The visual aid device may also have removable lenses, and temples or stems that may have lens mounts to store a lens. A blocking material may be used to produce regions or portions of visual aid device through which a user has no visibility.

### CROSS-REFERENCE TO RELATED APPLICATIONS

This application claims priority to Non-Provisional application Ser. No. 13/168,142 filed 24 Jun. 2011, with Non-Provisional application Ser. No. 13/168,142 incorporated by reference and whole or in part in the current application.

### FEDERALLY SPONSORED RESEARCH

Not applicable.

### BACKGROUND OF THE INVENTION

The present invention generally relates to sports training aids, and more particularly, to a visual aid device.

In some sports, an athlete's performance is dependent partially on focus and the ability to concentrate on the action being performed. The better one can maintain focus on a particular action or movement, the higher the likelihood, the athlete will succeed at it. Indeed, some athletes and coaches preach being able to mentally block out everything but the goal at hand.

For example, a baseball or softball player may spend countless hours swinging a round bat trying to hit a moving round ball hurled speeding at them; what some consider the most difficult feat in sports.

One of the obstacles that inhibits an athlete's success in an action is the distractions within a surrounding environment. For example, a common distraction in sports is a surrounding crowd attending the game or match. A crowd sitting in stands may actively attempt to distract an athlete by waving arms, towels, etc. in an attempt to momentarily catch the athlete's eye and distract him from an impending action (for example, hitting a pitch or shooting a basketball). The crowd or even other players may passively also serve as a distraction engaging in routine movement which is picked up by the athlete's field of view (for example, fans walking about, player's moving into position on a baseball diamond). The athlete's brain may subconsciously register this movement thus, distributing focus or concentration away from the task at hand. Additionally, some athletes my experience double vision or seeing double known as diplopia.

As can be seen, there is a need for device that can block distractions from the peripheral vision of an athlete and also another device that can be produced to limit the effects of diplopia.

### SUMMARY OF THE INVENTION

In one aspect of the present invention, a visual aid device comprises a frame and a lens coupled to the frame. The visual aid device also comprises an opaque upper portion of the lens, an opaque lower portion of the lens, and a transparent region juxtaposed between the opaque upper and lower portions of the lens.

In another aspect of the present invention, a visual aid device comprises a frame configured to mount in front of a user's eyes, one or more upper baffles coupled to the frame disposed to cover an upper peripheral vision along a horizontal plane above the user's eyes, and one or more lower baffles coupled to the frame disposed to cover a lower peripheral vision along a horizontal plane below the user's eyes, wherein a lower edge of the upper baffles and are disposed spaced from an upper edge of the lower baffles to define a tunnel vision field of view therebetween.

In yet another aspect of the present invention, a visual aid device comprises a frame and a lens coupled to the frame. The frame being a removable lens frame. The visual aid device may also comprise an opaque inner portion of the lens, and a transparent region on an outer portion the lens, the outer portion being on the opposite side of the removable lens from the bridge.

In still yet another aspect of the present invention, a visual aid device comprises a frame and a lens coupled to the frame. The frame being a removable lens frame. The visual aid device may also comprise an opaque inner portion of the lens. The opaque inner portion may a blocker that may be sized and shaped using a template. The visual aid device may further have stems or temples with lens mounts for storing lenses.

These and other features, aspects and advantages of the present invention will become better understood with reference to the following drawings, description and claims.

### BRIEF DESCRIPTION OF THE DRAWINGS

FIG. 1 is a frontal perspective view of a visual aid device according to an exemplary embodiment of the present invention;

FIG. 2 is a front view of the visual aid device of FIG. 1;

FIG. 3 is a side view of the visual aid device taken along the line 3 -3 of FIG. 2 illustrating a tunnel vision created by the device during use;

FIG. 4 is an illustration of an exemplary use of the visual aid device as worn by a user;

FIG. 5 shows blocking material and a template used to shape the material for application to a lens;

FIG. 5A is an enlarged view of the blocking material;

FIG. 6 is an enlarged view of the template;

FIG. 7 is a frontal view of a removable lens frame;

FIG. 7A is a top view of the removable lens frame;

FIG. 7B is a top sectional view along line 7B-7B of FIG. 7;

FIG. 7C is a sectional view along line 7C-7C of FIG. 7A showing lens tracks and tab holders;

FIG. 8 is a frontal view of the removable lens frame showing lenses ready for insertion in the other frame;

FIG. 9 is a sectional view along line 9-9 of FIG. 8 showing the lens track and the tab holders;

FIG. 10 is a sectional view along line 10-10 of FIG. 8 showing the lens track and the tab holders;

FIG. 11 is a frontal view of the removable lens frame with the lenses inserted;

FIG. 12 is a stem inner surface view of a stem front portion with a lens mount for caring lenses;

FIG. 13 is a sectional view along line 13-13 of FIG. 12 showing a lens mount with the tab holders;

FIG. 14 is a sectional view along line 14-14 of FIG. 12 showing a lens mount with the tab holder;

FIG. 15 is a stem inner surface view of a stem front portion with a modified lens mount for caring lenses;

FIG. 15A is a view of the modified lens mount along line 15A-15A of FIG. 15;

FIG. 15B is a view of the mount top side of the modified lens mount;

FIG. 15C is a view of the mount first side of the modified lens mount;

FIG. 16 is a view of several lens combinations that can be inserted in the removable lens frame;

FIG. 17 is a view of partial lenses, half lenses, separated by air that can be inserted in the removable lens frame; and

FIG. 18 is another view of the removable frame of lens frame with three tab holders in the lens track.8

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims. Various inventive features are described below that can each be used independently of one another or in combination with other features.

Broadly, embodiments of the present invention generally provide a visual aid device that can be used by, for example, an athlete (also sometimes referred to as a user in the following disclosure). The visual aid device may provide a user with an aid to increase focus by providing a tunnel vision to the user thus, enhancing a user's focus on a task at hand.

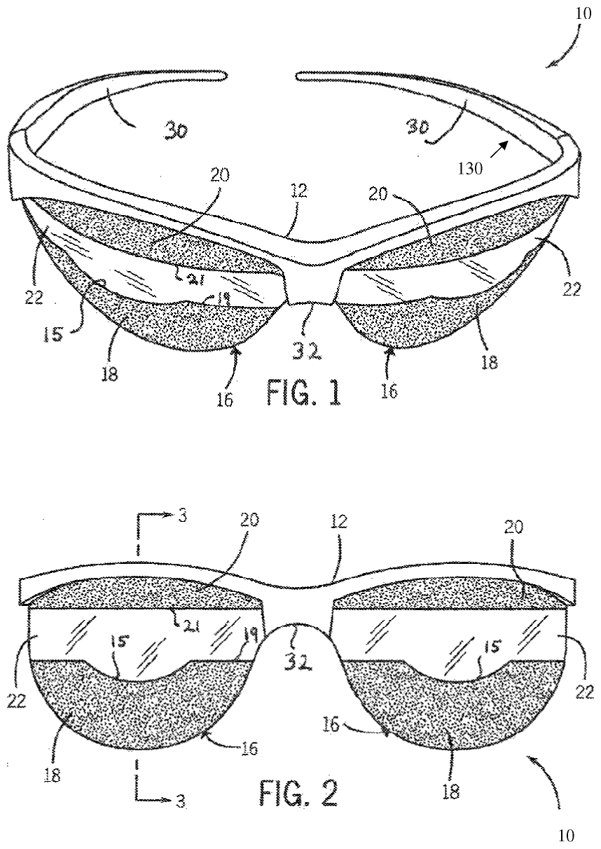

Referring to FIGS. 1-3 an exemplary embodiment of a visual aid device 10 is shown. The visual aid device 10 includes a frame 12, configured to mount in front of a user's eyes, with an opaque upper portion 20 disposed to cover an upper peripheral vision along a horizontal plane above the user's eyes and an opaque lower portion 18 disposed to cover a lower peripheral vision along a horizontal plane below the user's eyes. In one aspect, the upper and lower opaque regions (portions) 18 and 20 may block out light peripherally above and below the eyes.

In one exemplary embodiment, the visual aid device may be eyewear, for example, modified glasses. The visual aid device 10 may include one or more lenses 16, a bridge 32 holding the frame 12 onto a user's nose, and one or more temples 30 that may be tensioned to assist in holding the visual aid device 10 onto the user's head. The lenses 16 may be hard plastic or glass lenses. In one exemplary embodiment, the opaque upper portion 20 and the opaque lower portion 18 may be a dark tint integrated into the lens material. In another exemplary embodiment, the opaque upper portion 20 and the opaque lower portion 18 may be darkened hard plastic or soft vinyl covers affixed to either a rear surface 27 or front surface 23 of the lenses 16. In another exemplary embodiment, the frame 12 may not include lenses and the opaque upper portion 20 and the opaque lower portion 18 may be baffles attached to the frame 12.

The opaque upper portion 20 and the opaque lower portion 18 may be disposed spaced from one another. The lens 16 may include a transparent region 22 juxtaposed between the opaque upper portion 20 and the opaque lower portion 18. The transparent region 22 may extend laterally between one side edge of the lens to the opposite side edge. In exemplary embodiments that do not include a lens 16, the transparent region 22 may be open air. The height of the transparent region 22 and the spacing between the opaque upper portion 20 and the opaque lower portion 18, may be approximately the height of the user′ eye. For example, the opaque upper portion 20 may include a lower edge 21 positioned proximate to the top of and above the user's eye. The opaque lower portion 18 may include an upper edge 19 that may be positioned proximate to and below the bottom of the user's eye. While the transparent region 22 is shown with substantially parallel lower and upper edges 19 and 21, another exemplary embodiment may include lower and upper edges 19 and 21 that taper in narrowing fashion toward one another toward the outer edges of the lenses 16.

In one aspect, the visual aid device 10 may accommodate varying eye sizes by including a depression 15 along the upper edge 19. The depression 15 may provide an eye mark for proper positioning of the eyes while using the visual aid device 10. The depression 15 may also provide for an unobstructed field of vision just around the user's pupil in what is an otherwise obstructed view.

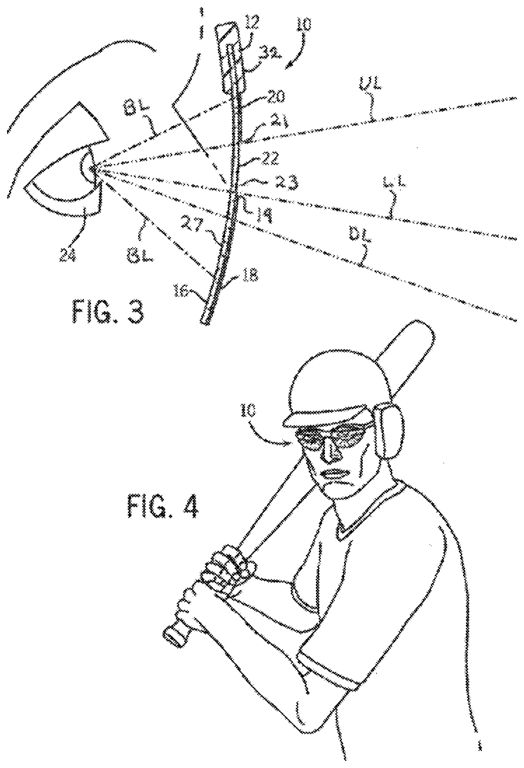

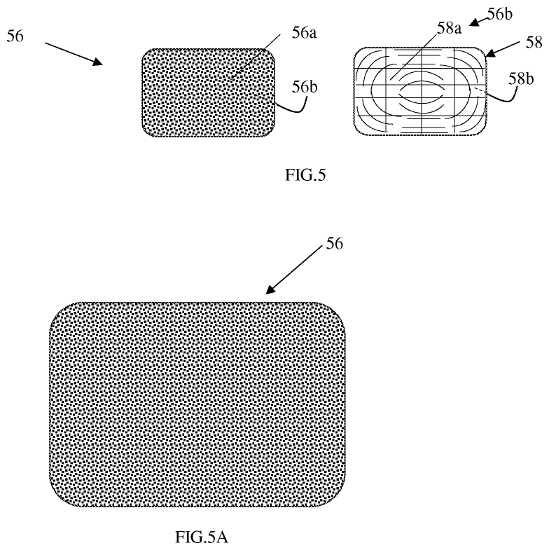

Referring now to FIGS. 3 and 4, an exemplary use of the visual aid device 10 is shown. A baseball player may wear the visual aid device 10 during the act of batting. The opaque upper portion 20 and the opaque lower portion 18 may limit the batter's field of view by blocking out light rays BL that are in horizontal planes above and below the user's eyes. The light rays UL and LL shown may represent boundaries of the horizontal planes above and below the user's eyes. Thus, the transparent region 22 may define a tunnel vision by allowing light rays between and including rays UL and LL under the lower edge 21 and above the upper edge 19 respectively to reach the user's eyes. Additionally, exemplary embodiments including the depression 15 may allow more light rays DL through the transparent region exposed by the depression 15. Thus, distractions that may have otherwise been detectable in the light rays BL are blocked from the user's sight while the user may be allowed to focus on, for example, a pitched ball that is viewable within the visual aid device 10 transparent region 22.

While the foregoing has been described in the context of use by a baseball batter, it will be understood that other athletes may benefit from the tunnel vision provided by the visual aid device 10. For example, a basketball player may employ the visual aid device 10 during shooting and a golfer may employ the visual aid device 10 during a golf swing.

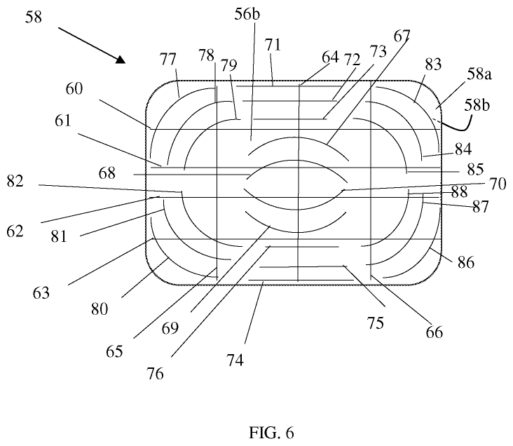

Referring to FIGS. 5, 5A, and 6, another visual aid device may include blocking material, a blocker 56, having a blocker one side 56a and a blocker opposite side 56b, that may be a cut-line template, or template 58 having a template one side 58a and a template opposite side 58b with one or more cut lines. Heavy fabrics, plastics, wood, and metal are some of the materials that may be used to make opaque blocking material. Referring to FIG. 6, the template 58, on the template one side 58a, there may have; first, second, third, and fourth horizontal cut lines 60,61,62, 63; first, second and third vertical cut lines 64,65; 66, first and second upper curved cut lines 67,68; first and second lower curved cut lines 69,70; first, second, and third upper horizontal cut lines 71, 72,73; first, second, and third lower cut line 74,75, 76; first, second, and third upper inner cut lines 77,78,79; first, second, and third lower inner cut lines 80,81,82; first, second, and third upper outer cut lines 83,84,85; and first, second, and third lower outer cut lines 86,87,88. The template 58 may be made of clear plastic allowing the user to cover the lens 16 and observe where to cut the blocker 56 for applying to the lens 16. The cut lines, such as the first, second, third, and fourth horizontal cut lines 60,61,62, 63 may slots through the template one side 58a to the template opposite side 58b, allowing the user to mark the intended cut lines on the blocker 56. The blocker 56 may be opaque with no transparency or have varying degrees of transparency depending on the desired application. The blocker 56 may be applied to conventional lenses available in the stream of commerce. The blocker 56 may be attached to the lenses 16 using adhesive or may be attached electrostatically. The blocker 56 may also integrated in the lens 16 of FIG. 1

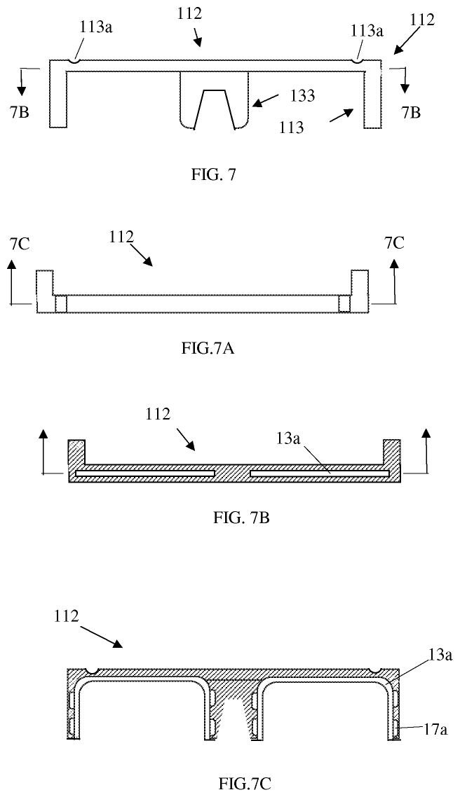

Referring to FIGS. 5, 7, 7A, 7B, 7C and FIG. 8, there may be a device kit 8 that may include the blocker 56, a template 58, a removable lens frame 112, removable lenses 116 that may have another transparent region 122, and the stems, also called temples 30 as seen in FIG. 1, to produce the other visual aid device. The removable lens frame 112 may have a frame extension 113 opposite a bridge extension 133 and a compression points 113a to relieve stress when the frame extension 113 is extended away from the bridge extension 133 for installing and removing a lens. Referring again to FIG. 7C, the removable lens frame 112 may further have one or more lens tracks 13a for the lens to lay in and tab holders 17a to hold the lens in the frame.

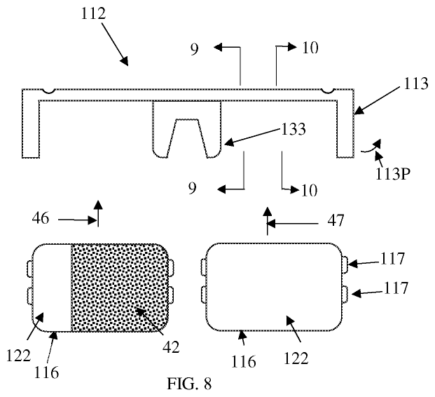

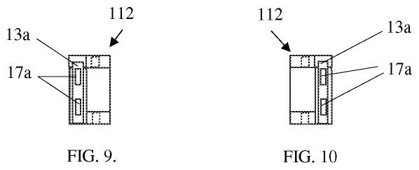

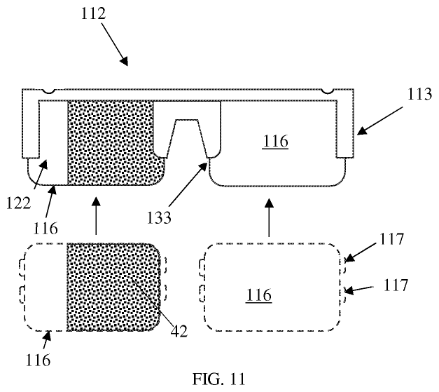

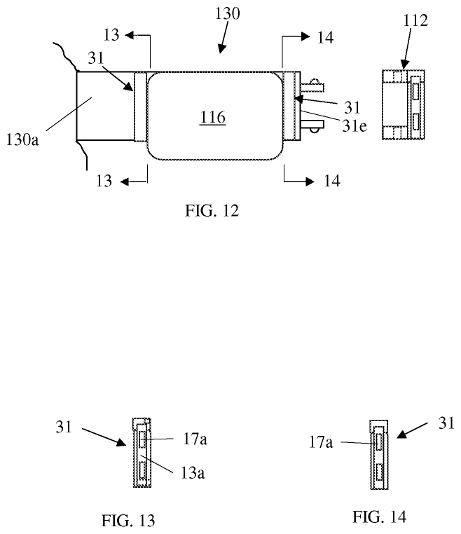

Referring to FIGS. 7C and 8, the frame extension 113 may move along swing path 113p to install and remove a removable lens 116. Illustrated is a removable lens 116 with and opaque inner portion 42 and a removable lens 116 without the blocker. This configuration is ideal for some people experiencing double vision, The removable lenses 116 will be move along lens paths 46,47 with the lens tabs 117 sliding in the lens track 13a of FIGS. 7C, 9 and 10 and resting in the tab holders 17a, secured in place once the frame extension 113 is released and moves back toward the bridge extension 133. FIG. 11 is a frontal view of the removable lens frame 112 with the removable lenses 116 inserted. Referring to FIGS. 12, 13, and 14, each stem or temple 30 may have two lens mounts 31 for storing lenses. The lens mounts 31 may be on a stem inner surface 130a of a stem front portion 130. A first lens mount 31 may be located to the rear of the stem connect end 31e with the second lens mount 31 located to the rear of the first lens mount 31. Each of the lens mounts 31 may have one or more tab holders 17a that may be in the same configuration as the tab holders 17a on the removable lens frame 112 and/or the frame extension 113. The stem forward portion 130 may be may flexed to make a stem inner surface 130a convex, allowing the lens tabs 117 shown in FIG. 8 to be inserted in the tab holders 17a of the lens mounts 31.

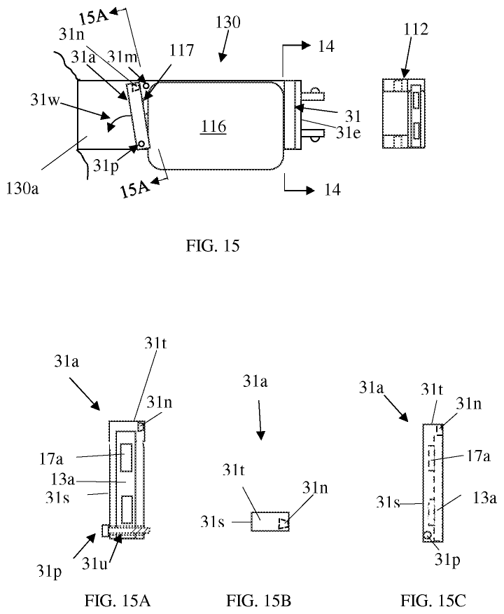

Referring to FIGS. 15, 15A, 15B, and 15C, the second lens mount 31 may be a modified lens mount 31a that rotates along a mount path 31w about a mount pin 31p located in a pin shaft 31u. The removable lens 116 is mounted in the first lens mount 31 and the modified lens mount 31a rotated back to lock mount 31m with the lock mount 31m secured in a mount holder 31 n. FIG. 15A is view along line 15A-15A of FIG. 15 showing the mount pin 31p positioned in the pin shaft 31u through a mount first side 31s. FIG. 15B is view of the modified mount 31a from the mount top side 31t. FIG. 31 C is a view of the modified mount 31a from the mount first side 31s.

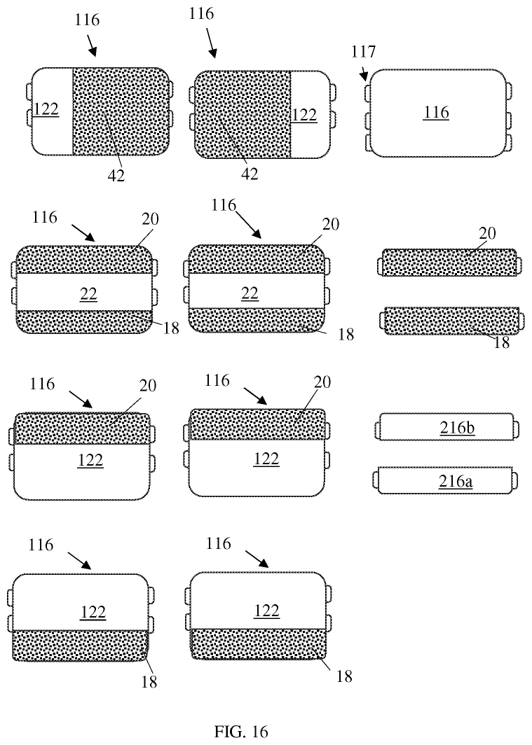

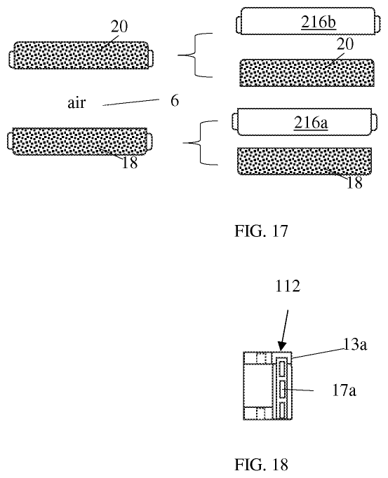

FIG. 16 shows a variety of removable lenses 116, some with and some without opaque inner portions 42, opaque upper portions 20 and opaque lower portions 18. The opaque inner, upper, and lower portions 41,20, 18, may constructed with the blocker 56 of FIG. 6 applied to the removable lens 116 or the upper and lower half lens 216b, 216a. There are a variety of configurations shown: an opaque lower portion 18 and opaque upper portion 20 with a transparent region 22 there between; some with just the opaque upper portion 20, some just the opaque lower portion 18; a removable lens 116 with three lens tabs 117; a removable lens 116 with an opaque inner portion 42; and an upper half lens 216b and a lower half lens 216a, a pair with and a pair without the blocker 56. Referring to FIG. 17, an illustration of an opaque lower portion 20 separated from an opaque upper opaque upper portion 20 with air 6 therebetween is shown. FIG. 18 shows a lens track 13a of the removable lens frame 112 with three tab holders 17a. Referring to FIGS. 17 and 18, a upper half lens 216b could be mounted in the one tab holder 17a and a lower half lens 216a can mounted in another tab holder with a center tab holder 17a empty allowing for the air 6 there between for the configuration of FIG. 17.

It should be understood, of course, that the foregoing relates to exemplary embodiments of the invention and that modifications may be made without departing from the spirit and scope of the invention as set forth in the following claims.

What is claimed is:

1. A visual aid device further comprising:
a frame;
a lens coupled to the frame;
an opaque upper portion of the lens;
an opaque lower portion of the lens; and
a transparent region juxtaposed between the opaque upper
and lower portions of the lens.

2. The visual aid device of claim 1, further comprising a depression in the lower opaque portion of the lens.

3. The visual aid device of claim 2, wherein the depression is disposed in front of a user's pupil when the visual aid device is worn.

4. The visual aid device of claim 1, further comprising at least one temple configured to fit the visual aid device to a user's head.

5. A visual aid device comprising:
a frame configured to mount in front of a user's eyes;
one or more upper baffles coupled to the frame disposed to cover an upper peripheral vision along a horizontal plane above the user's eyes; and
one or more lower baffles coupled to the frame disposed to cover a lower peripheral vision along another horizontal plane below the user's eyes, wherein a lower edge of the upper baffles and are disposed spaced from an upper edge of the lower baffles to define a tunnel vision field of view therebetween.

6. The visual aid device of claim 5, further comprising a depression formed on the upper edge of the lower baffle.

7. The visual aid device of claim 5, wherein lower edge of the one or more upper baffles is configured for position above the user's eyes.

8. The visual aid device of claim 5, wherein the one or more upper and lower baffles are integrated into a pair of lenses.

9. The visual aid device of claim 5, wherein the one or more upper and lower baffles are affixed to, either in front of or behind, one or more lenses mounted on the frame.

10. The visual aid device of claim 9, wherein the baffles are plastic covers affixed to the lenses.

11. A visual aid device comprising:
a removable lens frame;
a removable lens coupled to the removable lens frame;
and an opaque inner portion.

12. The visual aid device of claim 11, wherein the removable lens frame has at least one frame extension opposite a bridge extension.

13. The visual aid device of claim 12, wherein the removable lens has at least one lens tab.

14. The visual aid device of claim 13, wherein the removable lens frame has at least one lens track.

15. The visual aid device of claim 14, wherein the lens track has at least one tab holder.

16. The visual aid device of claim 15, wherein the removable lens frame has at least one compression point.

17. The visual aid device of claim 11, further comprising at least one temple to fit the visual aid device to a user's head.

18. The visual aid device of claim 17 wherein the temple of claim further comprises two lens mounts; and the lens mounts further comprising a lens track and at least one tab holder.

19. The visual aid device of claim 17 wherein the temple further comprises a lens mount and a modified lens mount; the modified lens mount and the lens mount further comprising a lens track and at least one tab holder; and the modified lens mount capable of rotation about a mount pin.

20. A visual aid device that is a visual focus kit comprising: a frame to mount in front a user's eyes; a blocking material to block light; a template with cut lines; and two or more lenses for the frame.
